(19) United States
(12) Patent Application Publication
(10) Pub. No.: US 2008/0167592 A1
(43) Pub. Date: Jul. 10, 2008

(54) Preventing or treating wounds with a collodion barrier incorporating active agents

(76) Inventor: Steven E. Greer, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/977,813

(22) Filed: Oct. 26, 2007

Related U.S. Application Data
(60) Provisional application No. 60/854,511, filed on Oct. 26, 2006.

(51) Int. Cl. A61L 26/00; A61F 13/00

(52) U.S. Cl. 602/43; 602/50; 602/48

(57) ABSTRACT

A method of inhibiting and/or treating wounds on a patient's skin includes applying a covering of collodion over an area on the patient's skin which is prone to formation of an ulcer or to a wound on the patient's skin where a wound has formed. The covering of collodion may contain an active ingredient to stimulate healing of the area covered by the collodion.

### BACKGROUND OF THE INVENTION

The present invention is directed to a method of inhibiting and/or treating of pressure ulcers, skin breakdown, or abrasions, heretofore collectively referred to as “wounds” on a patient's skin.

It has previously been suggested that cyanoacrylate adhesives may be used to inhibit skin ulceration and/or to treat skin ulceration. The skin ulceration may be pressure ulcers, (AKA bed wounds or decubitus ulcers). Alternatively, the skin ulcerations may be diabetic ulcers. A method of using cyanoacrylate adhesive to inhibit and/or treat ulcers is disclosed in U.S. Pat. No. 6,492,434.

### SUMMARY OF THE INVENTION

The present invention relates to a new and improved method of inhibiting and/or treating wounds on a patient's skin. The method includes locating a treatment area on the patient's skin which is prone to formation of a wound or a treatment area on the patient's skin where a wound has formed. A covering of collodion is applied over the treatment area.

It is contemplated that the covering of collodion may contain an active ingredient to stimulate healing of skin covered by the collodion. When the covering of collodion is applied over a wound, the collodion and active ingredient(s) will reduce inflammation and pain, and will stimulate healing of the wound. Although the active ingredient is advantageously applied to the skin on a patient's body with a covering of collodion, it is contemplated that the covering of collodion and the active ingredient may be applied to tissue on a patient's body other than the skin.

It is contemplated that the active ingredient which is contained in the covering of collodion may be a growth factor, an antibiotic, or an antifungal agent. The growth factor may be a fibroblast growth factor. As one example, the growth factor may be a keratinocyte growth factor. The active ingredient may be a recombinant human platelet-derived growth factor, such as becaplermin. There are many known growth factors which may be utilized, including polypeptides and vascular endothelial growth factors.

### BRIEF DESCRIPTION OF THE DRAWINGS

The foregoing and other features of the invention will become more apparent upon consideration of the following description taken in connection with the accompanying drawings wherein:

FIG. 1 is a schematic illustration depicting the manner in which a covering of collodion is provided over a treatment area on a patient's skin.

### DESCRIPTION OF SPECIFIC PREFERRED EMBODIMENTS OF THE INVENTION

The present invention relates to the method of inhibiting and/or treating wounds on a patient's skin. The wounds may be at a location where the skin is abraded and/or ruptured. The wounds may or may not be infected. The wounds may have been created by cosmetic surgery. The wounds may be ulcers. It is contemplated that the method of the present invention may be utilized in association with soft tissue at many different locations in a patient's body.

The patient's body is examined to locate a treatment area on the patient's skin. The treatment area may be an area which was abraded by cosmetic surgery. The treatment area may be an area which is prone to formation of an ulcer or other wound. Alternatively, the treatment area may be an area on the patient's skin where an ulcer or other wound has formed.

The wound may be an ulcer; a donor wound site wound; a surgical wound; a wound made during cosmetic surgery; a dermal wound; an excisional wound; a wound involving damage of the dermis and/or epidermis; an anastomosis; a burn; a wound from ischemia; and tissue trauma. The wound may be due to any one or more of many conditions, including diabetes, ischemic blockage, uremia, malnutrition, vitamin deficiency, obesity, infection, immunosuppression, and radiation therapy.

When the wound is an ulcer, the ulcer may be a stage 1 ulcer characterized by redness of the skin or a stage 2 ulcer which is similar to an abrasion of the skin. The ulcer may be a stage 3 ulcer which goes through the skin. It is believed that the covering of collodion will be particularly advantageous in the treatment of stage 1 and stage 2 ulcers. It is also believed that the covering of collodion will be applied to treatment areas on a patient's skin at locations which are prone to the formation of ulcers and/or other wounds due to the conditions to which the skin is exposed.

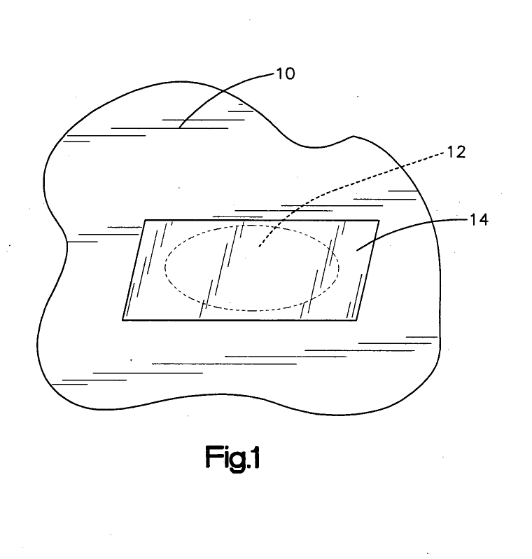

The patient's skin 10 has been illustrated schematically in FIG. 1. A treatment area 12 on the patient's skin has been indicated schematically in dashed lines in FIG. 1. As was previously mentioned, the treatment area 12 may be an area on the patient's skin which is prone to formation of a wound or an area on the patient's skin where a wound has formed. It is believed that the present invention will be particularly advantageous in the treatment of wounds which are ulcers. The ulcer may or may not be infected.

Assuming that an ulcer has formed or has started to form at the treatment area 12, the ulcer may be a pressure ulcer. The ulcer at the treatment area 12 may be at any one of different stages of development, such as a stage 1, stage 2 or stage 3. Of course, if the treatment area 12 is an area which is only prone to the development of an ulcer, there may be no ulcer at the treatment area.

In accordance with one of the features of the present invention, a covering 14 of collodion is applied over the treatment area. The flexible covering of collodion reduces the coefficient of friction on the skin 10 at the treatment area 12. Assuming that the treatment area 12 is an area which is prone to the development of an ulcer, the covering 14 of collodion will reduce the risk factor for development of a pressure ulcer by reducing friction. In addition, covering 14 will help to prevent breakdown of the skin 10 at the treatment area 12 to further prevent the development of an ulcer.

The covering 14 forms a waterproof barrier that protects the treatment area 12 from moisture. With an incontinent patient, the covering 14 of collodion will prevent exposure of the treatment area 12 to urinary and/or fecal materials to thereby reduce the possibility of forming an ulcer. The covering of collodion is thicker and sturdier than cynoacrylate coverings. The covering of collodion is a slick friction reducing surface that is a moisture barrier. The covering of collodion has an outer side surface with a lower coefficient of friction than a covering of cyanoacrylate. A covering of collodion is sturdier and is a better friction reduction barrier than a covering of cyanoacrylate.

If an ulcer has already formed at the treatment area 12, the flexible transparent covering 14 provides sturdy a barrier which prevents abrasion of the skin at the treatment area. Thus, the smooth covering 14 prevents abrasion of the treatment area 12 by engagement with sheets on a bed in which a patient is disposed. Of course, the smooth flexible covering 14 may be used to protect the treatment area 12 against exposure to the anything in the environment around the patient, such as plastic tubing that rests over the ears and conducts a flow of oxygen. The covering 14 prevents a stage 1 ulcer from developing into a stage 2 or stage 3 ulcer. Similarly, the covering prevents a stage 2 ulcer from developing into a stage 3 ulcer. If a stage 3 ulcer is present at the treatment area 12, the covering 14 protects the ulcer from exposure to the environment around the patient and promotes healing of the ulcer.

The flexible smooth covering 14 is formed of collodion. Collodion is a colorless syrupy solution of proxylin in ether, acetone, or alcohol. The collodion has a viscosity which promotes solidification when it is applied over the treatment area 12 on the skin 10 to form a flexible covering 14 which is transparent. Castor oil can be added to make it more flexible. The covering 14 of collodion may be similar to the coverings disclosed in U.S. Pat. Nos. 4,681,635 and/or 5,433,950. The disclosures in the aforementioned U.S. Pat. Nos. 4,681,635 and 5,433,950 are hereby incorporated herein by this reference thereto.

When the collodion has dried to form the flexible transparent covering 14, the treatment area 12 is protected against engagement with either solids or liquids in the environment to which the skin 10 of the patient is exposed. The covering 14 of collodion is waterproof so that liquids cannot seep through the covering to the treatment area 12. It is believed that the waterproof aspect of the covering 14 of collodion will be particularly advantageous when a patient is incontinent. The treatment area 12 is visible through the flexible covering 14 of collodion. This enables the treatment area 12 to be visually examined with the covering 14 in place.

In accordance with another feature of the present invention, the flexible transparent covering 14 of collodion contains an active ingredient to stimulate healing of the treatment area 12. The active ingredient is mixed into the colorless syrupy solution of collodion and is applied to the treatment area simultaneously with the covering 14 of collodion. If desired, a coating of the active ingredient may be applied to the treatment area 12 prior to application of the flexible transparent covering 14 of collodion over the treatment area. The active ingredient which is mixed with the clear colorless syrupy collodion solution may be the same active ingredient as is applied over the treatment area 12 separately from the collodion or may be a different active ingredient.

The active ingredient, contained in the covering 14 of collodion may be any one of many known different ingredients which promote healing of the treatment area 12 and which retard any tendency for the formation of a wound, such as an ulcer, at the treatment area 12 The active ingredient may be a fibroblast growth factor. Specifically, the active ingredient may be a keratinocyte growth factor which is sometimes referred to as “KGF” and also “KGF-2”.

The active ingredient may be vascular endothelial growth factor which is sometimes referred to as “VEGF” and “VEGF-2”. The active ingredient may be a combination of many different ingredients which promote healing. The active ingredient may be becaplermin which is also recombinant human platelet-derived growth factor (rhPDGF-BB) such as Johnson and Johnson's “Regranex”™.

The active ingredient in the clear flexible covering 14 of collodion may be becaplermin, a recombinant human platelet-derived growth factor (rhPDGF-BB) for topical administration. The active ingredient in the covering 14 of collodion may be an antibiotic such as quinolone. An antifungal agent may be provided in the covering 14 of collodion. It is contemplated that each of the aforementioned active ingredients may be utilized by itself or in combination with one or more other active ingredients. Of course, various combinations of many known active ingredients which promote healing of a wound covered by the covering 14 of collodion may be provided in the collodion.

The active ingredient may be a polynucleotide, polypeptide encoded by a polynucleotide. Polynucleotides and other known substances may be utilized to accelerate healing of a wound, such as an ulcer, at the treatment area 12. Tissue growth factors may be applied directly to the treatment area 12 and/or may be provided in the covering 14 of collodion. The tissue growth factor applied to the treatment area 12 may be the same as the growth factor contained in the covering 14 of collodion. Alternatively, the growth factor applied directly to the treatment area 12 may be different than the growth factor contained in the covering 14 of collodion. Of course, a plurality of different growth factors may be provided in the covering 14 of collodion and/or may be applied directly to the treatment area 12.

The growth factors which are utilized may be similar to any one of the growth factors described in U.S. Pat. No. 6,693,077. The disclosure in U.S. Pat. No. 6,693,077 is hereby incorporated herein in its entirety by this reference thereto The growth factors which are utilized may be similar to any one of the growth factors described in the U.S. Pat. No. 6,045,565. The disclosure in the U.S. Pat. No. 6,045,565 is hereby incorporated herein in its entirety by this reference thereto.

The active ingredient mixed with the collodion is gradually exposed to the treatment area 12. A portion of the active ingredient mixed with the collodion in the covering 14 is immediately exposed to the treatment area 12. With the passage of time, the collodion on the side of the covering 14 toward the treatment area 12 enrodes. This results in exposure of additional active ingredient in the covering 14 and release to the treatment area 12.

The amount of the active ingredient contained in the collodion forming the transparent flexible covering 14 will determine the rate at which the treatment area 12 is exposed to the active ingredient. The greater the amount of active ingredient contained in a predetermined volume of collodion, the greater will be the rate at which the active ingredient is released from the transparent covering 14 and the greater will be the rate of exposure of the treatment area 12 to the active ingredient. The concentration of the active ingredient in the collodion forming the covering 14 can be varied to provide any desired rate of release of the active ingredient with the passage of time.

Although it is believed that it may be preferred to provide an active ingredient in the collodion forming the covering 14, the covering may be devoid of active ingredients other than collodion. A flexible covering 14 of collodion which is free of active ingredients will still promote healing of any wounds at the treatment area 12. If the skin 10 at the treatment area 12 is merely abraded or inflamed, a covering 14 of collodion without active ingredients would protect the treatment are from engagement with sheets or other bedding. The transparent flexible covering 14 of collodion prevents exposure of the treatment area 12 to the atmosphere.

In view of the foregoing description, it is clear that the present invention provides a new and improved method of inhibiting and/or treating wounds on a patient's skin 10. The method includes locating a treatment area 12 on the patient's skin 12 which is prone to formation of a wound or a treatment area on the patient's skin where a wound has formed. A covering 14 of collodion is applied over the treatment area.

It is contemplated that the covering 14 of collodion may contain an active ingredient to stimulate healing of skin 12 covered by the collodion. When the covering 14 of collodion is applied over a wound, the active ingredient in the covering of collodion will stimulate healing of the wound. Although the active ingredient is advantageously applied to the skin 10 on a patient's body with a covering 14 of collodion, it is contemplated that the covering of collodion and the active ingredient may be applied to tissue on a patient's body other than the skin.

It is contemplated that the active ingredient which is contained in the covering 14 of collodion may be a growth factor, an antibiotic, or an antifungal agent. The growth factor may be a fibroblast growth factor. As one example, the growth factor may be a keratinocyte growth factor. The active ingredient may be a recombinant human platelet-derived growth factor, such as becaplermin. There are many known growth factors which may be utilized including polypeptides.

Having described the invention, the following is claimed:

1. A method of preventions and/or treating wounds on a patient's skin, said method comprising the steps of locating a treatment area on the patient's skin which is prone to formation of a wound or a treatment area on the patient's skin where a wound has formed, and applying a covering of collodion over the treatment area.

2. A method as set forth in claim 1 wherein the covering of collodion contains an active ingredient to stimulate healing of any wound covered by the collodion.

3. A method as set forth in claim 2 wherein the active ingredient is a keratinocyte growth factor.

4. A method as set forth in claim 2 wherein the active ingredient is becaplermin.

5. A method as set forth in claim 2 wherein the active ingredient is an antibiotic.

6. A method as set forth in claim 2 wherein the active ingredient is an antifungal agent.

7. A method as set forth in claim 2 wherein the active ingredient is a fibroblast growth factor.

8. A method as set forth in claim 2 wherein the active ingredient is polypeptide.

9. A method as set forth in claim 1 wherein said step of locating a treatment area on the patient's skin where a wound has formed includes locating an area on the patient's skin where a stage 2 ulcer has formed.

10. A method of preventing and/or treating wounds on a patient's body, said method comprising the steps of locating a treatment area on the patient's body, and applying a covering of collodion over the treatment area, wherein the patient a has a wound selected from the group consisting of:
(a) an ulcer;
(b) a donor site wound;
(c) a surgical wound;
(d) a wound made during cosmetic surgery;
(e) a dermal wound;
(f) an excisional wound;
(g) a wound involving damage of the dermis and/or epidermis;
(h) an anastomosis;
(i) a burn;
(j) a wound from ischemia; and
(k) tissue trauma.

11. A method as set forth in claim 12 wherein the wound is due to a condition selected from the group consisting of diabetes, ischemic blockage, uremia, malnutrition, vitamin deficiency, obesity, infection, immunosuppression, and radiation therapy.

12. A method of preventing and/or treating wounds on a patient's skin, said method comprising the steps of applying a covering of collodion over a treatment area on the patient's skin, wherein the covering of collodion is sturdier and is better friction reduction barrier than cyanoacrylate.
